(19) United States
(12) Patent Application Publication
(10) Pub. No.: US 2025/0050476 A1
(43) Pub. Date: Feb. 13, 2025

(54) SCREWDRIVER FOR AN INNER PROFILE SCREW

(71) Applicant: Peter WITTE, Kiel (DE)

(72) Inventor: Peter WITTE, Kiel (DE)

(21) Appl. No.: 18/763,614

(22) Filed: Jul. 3, 2024

(30) Foreign Application Priority Data
Aug. 11, 2023 (DE) 10 2023 121 570.1

(51) Int. Cl. B25B 23/10; A61B 17/88

(52) U.S. Cl. CPC B25B 23/108; A61B 17/888; A61B 17/8888

(57) ABSTRACT

A screwdriver (2) for an inner profile screw (50). The screwdriver includes a shaft (4) and an outer profile head (6) which is arranged at a distal end (7) of the screwdriver. The outer profile head includes a gap (10) along the longitudinal axis which splits the outer profile head into a large profile head part (30) and a small profile head part (20). A surface area of an end profile (31) of the large profile head part is greater than a surface area of an end profile (21) of the small profile head part. The end profiles of the large and small profile head parts in each case include multiple radially outwardly protruding teeth (22, 32). An outer radius (25) of the end profile of the small profile head part is greater than an outer radius (35) of the end profile of the large profile head part.

### PRIORITY CLAIM

This application claims priority to DE 10 2023 121 570.1, filed Aug. 11, 2023.

### BACKGROUND OF INVENTION

### Field of Invention

The invention relates to a screwdriver for an inner profile screw.

### Brief Description of Related Art

When surgically introducing an implant into the body of a person, inner profile screws are often used, which are tightened by means of a specially provided screwdriver. For example, when implanting a splint to be screwed to a bone, polygonal screws are typically used, which have a polygonal inner recess as the screw head drive. The outer profile head of the screwdriver is specially adapted to the inner profile recess in order to securely screw in the polygonal screw.

In order to prevent the screw from coming loose from the screwdriver during insertion of the implant, it is often provided to fix the outer profile head of the screw in the screw head drive such that the screw cannot inadvertently fall off. This can be done, for example, by means of clamping and/or magnetic force.

WO 2007/006282 A2 discloses a screwdriver for an inner profile screw, wherein the outer profile head is split into two parts. By moving a sleeve relative to a shaft of the screwdriver, the two parts of the outer profile head are sheered away from each other such that they become clamped in the inner profile recess of the screw.

It is an object of the invention to provide a screwdriver for an inner profile screw which simultaneously offers a high holding force and high strength with little wear of the outer profile head.

### BRIEF SUMMARY OF THE INVENTION

This object is achieved by a screwdriver for an inner profile screw, comprising a shaft and an outer profile head which is arranged at a distal end of the screwdriver, wherein the outer profile head comprises a gap along a longitudinal axis which splits the outer profile head into a large profile head part and a small profile head part, wherein a surface area of an end profile of the large profile head part is greater than a surface area of an end profile of the small profile head part, wherein the end profile of the large profile head part and the end profile of the small profile head part in each case comprise multiple radially outwardly protruding teeth, wherein an outer radius of the end profile of the small profile head part is greater than an outer radius of the end profile of the large profile head part.

The outer profile head of the screwdriver is thus split into a large profile head part and a small profile head part, the end profiles of which in each case comprise multiple outwardly protruding teeth. Due to the fact that the outer radius of the end profile of the small profile head part is greater than the outer radius of the end profile of the large profile head part, the two profile head parts can have different functions. Due to the fact that the end profile of the small profile head part projects further outward than the end profile of the large profile head part, the holding forces between the inner profile recess of the screw, on the one hand, and the outer profile head of the screwdriver, on the other hand, are increased. The small profile head part thus primarily serves to clamp the outer profile head in the inner profile recess of the screw. Due to the fact that the end profile of the large profile head part projects outward less far than the end profile of the small profile head part, the strength of the outer profile head is improved, since less wear is produced at high torques. This is based on the finding that, when tightening or loosening inner profile screws, the greatest wear occurs on the radially outermost parts of the outer profile head and the inner profile recess. In order to better absorb the torques, the surface area of the end profile of the large profile head part is greater than the surface area of the end profile of the small profile head part. Thus, due to the outer profile head designed according to the invention, reduced wear and a high torque are achieved when introducing the screw.

When viewed from the front along the longitudinal axis, the end profile of the outer profile head is split into the end profile of the large profile head part and the end profile of the small profile head part. Splitting the outer profile head makes it possible to fix the screwdriver in a screw drive of an inner profile screw. The outer profile head is adapted to the screw head drive of the inner profile screw which is to be screwed in by means of the screwdriver. For example, the outer head profile is a lobe (5-lobe, 6-lobe, 7-lobe, etc.) profile, for example a TORX® or TORX PLUS@geometry, a spline or SIT™ profile or another profile that is typically used for inner profile screws. The SIT™ profile is a brand used by Adolf Würth GmbH & Co. KG. The TORX® or TORX PLUS® are registered trademarks of Acument Intellectual Properties, LLC.

When the longitudinal axis is mentioned, this relates to a longitudinal axis of the screwdriver from the proximal end of the screwdriver to the distal end of the screwdriver or vice versa. The outer radius of the end profiles relates to the radius of a circular arc that is applied to the outer edges of the respective end profiles. Said circular arcs intersect, in particular, the tips of the teeth of the profile head parts.

Preferably, the teeth of the end profile of the large profile head part protrude less far radially outward than the teeth of the end profile of the small profile head part. The radial direction relates, in particular, to a center axis through the outer profile head in the longitudinal direction. In particular, a radius of a profile base from which the teeth of the end profiles protrude is identical for the small profile head part and for the large profile head part. Thus, in particular, only the shape of the outwardly protruding teeth of the profile head parts differs. In particular, the teeth of the end profile of the large profile head part are flattened.

Preferably, the teeth of the end profile of the small profile head part have rounded tips. Rounded tips are advantageous, since they allow for high holding forces with simultaneously reduced wear of the teeth. In particular, the teeth of the end profile of the small profile head part are completely rounded. In particular, the teeth of the end profile of the small profile head part transmit no torque when a screw is being screwed in or out. Instead, the torque is transmitted, in particular completely, through the teeth of the end profile of the large profile head part.

Preferably, the end profile of the large profile head part comprises more teeth than the end profile of the small profile head part. A larger number of teeth of the large profile head part is advantageous, since the large profile head part is mainly responsible for transmitting the torque. The exact number of teeth of the two end profiles depends on the inner profile recess of the inner profile screw for which the screwdriver is provided. In the case of inner profile screws having six recesses, the small profile head part has, for example, two teeth and the large profile head part has, for example, four teeth.

According to one embodiment, the end profile of the small profile head part and/or the end profile of the large profile head part is a star-shaped undulating profile. A star-shaped undulating profile of this kind is, for example, the profile known under the brand name “TORX® or TORX PLUS®”. For example, the end profile of the small profile head part is a TORX® profile and the end profile of the large profile head part is a TORX PLUS® profile.

Preferably, the end profile of the small profile head part and/or the end profile of the large profile head part in each case comprise, between the teeth, outer profile edges that have the shape of a depression or an outwardly curved circular arc. In particular, the teeth do not directly adjoin one another, but rather are separated from one another by means of the outer profile edges.

Preferably, a circular arc through the points lying in each case centrally between two teeth on the outer profile edge of the small profile head part has the same circle radius as a circular arc through the points lying in each case centrally between two teeth on the outer profile edge of the large profile head part. In other words, the base parts of the large profile head part and of the small profile head part, from which the teeth protrude, have the same outer radius. This is advantageous, since it makes it easier to hold and turn the inner profile screw by means of the two profile head parts.

Preferably, the end profile of the small profile head part and the end profile of the large profile head part have the same key size, such that they are designed for screwing in screws with screw head drives of the same size. In this way, the two profile head parts grip well in the screw head drive of the screw. In particular, the end profile of the profile head part has a geometric flattening. On account of this geometric flattening, the outer profile head is universally applicable.

A cleavage plane of the gap extends preferably in a longitudinal direction of the shaft and in a direction parallel to the end profile of the profile head parts. The gap thus separates the end profile of the outer profile head into the two profile head parts and extends from the end profile in a longitudinal direction in the direction of a screwdriver holder of the screwdriver.

Preferably, the gap splits the end profile of the outer profile head in an asymmetrical manner. In particular, the gap extends in the end profile of the outer profile head through a line of intersection that extends so as to be offset parallel to a center line through the end profile of the outer profile head.

According to one embodiment, the screwdriver comprises a clamping mechanism which is designed to sheer the small profile head part and the large profile head part relative to one another, wherein, in particular, a sheering direction extends along the gap in parallel with the end profiles of the profile head parts. By sheering the profile head parts relative to one another, they are clamped in the screw head drive of the inner profile screw. The small profile head part thereby ensures a secure hold in the screw head drive, whereas the large profile head part mainly absorbs the torques.

Preferably, the screwdriver comprises a sleeve that receives the outer profile head and that can be screwed in with the outer profile head. According to one embodiment, the outer profile head tapers toward the distal end in order to form at least one run-on slope, wherein the sleeve comprises an inwardly protruding run-on body which is designed to run on the at least one run-up slope in order to sheer the small profile head part and the large profile head part relative to one another. The run-on body is designed, in particular, as a circumferential run-on ring. Therefore, the sleeve with the run-on body as well as the run-on slope form the clamping mechanism, in particular. This manner of clamping the outer profile head in the screw head drive constitutes a reliable, reproducible, and user-friendly option for securing the outer profile head in the screw head drive.

Further features of the invention will become evident from the description of embodiments according to the invention, together with the claims and the appended drawings. Embodiments according to the invention can fulfill individual features or a combination of multiple features.

Within the framework of the invention, features which are labeled with “in particular” or “preferably” are to be understood to be optional features.

### BRIEF DESCRIPTION OF THE DRAWINGS

The invention will be described below without restricting the general concept of the invention by means of exemplary embodiments with reference to the drawings, wherein reference is expressly made to the drawings regarding all of the details according to the invention which are not explained in greater detail in the text, wherein:

FIG. 1 shows a schematically simplified perspective representation of a screwdriver for inner profile screws for surgical applications,

FIG. 2 shows a schematically simplified perspective representation of an outer profile head of the screwdriver in an untensioned state,

FIG. 3 shows a schematically simplified perspective representation of the outer profile head from FIG. 2 in a tensioned state,

FIG. 4 shows a schematically simplified cross-sectional representation through the outer profile head in the tensioned state, while the outer profile head is arranged in a screw head drive of an inner profile screw,

FIG. 5 shows a schematically simplified perspective representation through the outer profile head from FIG. 4 in the tensioned state,

FIG. 6 shows a schematically simplified perspective representation of the outer profile head in the untensioned state, wherein part of the sleeve has been omitted for better visibility of the inner components,

FIG. 7 shows a schematically simplified perspective representation of the outer profile head from FIG. 6 in the tensioned state,

FIG. 8 shows a schematically simplified cross-sectional view onto the outer profile head from FIG. 6 in the untensioned state, and

FIG. 9 shows a schematically simplified cross-sectional view onto the outer profile head from FIG. 7 in the tensioned state.

In the drawings, the same or similar elements and/or parts are, in each case, provided with the same reference signs, and therefore they are not introduced again in each case.

### DETAILED DESCRIPTION OF THE INVENTION

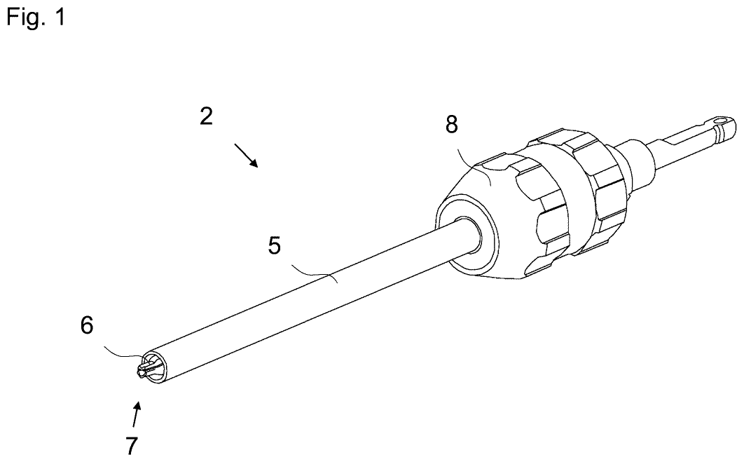

FIG. 1 shows, schematically simplified, a screwdriver 2 in a perspective representation. The screwdriver 2 comprises a rotatable handle 8, from which a shaft extends and opens out in an outer profile head 6 at the distal end 7 of the screwdriver 2. The shaft is enclosed by a sleeve 5, which conceals the shaft in FIG. 1. Screwdrivers 2 of this kind are used when surgically introducing implants into the body of a person in order to tighten or loosen inner profile screws in the implant. It is very important here that the inner profile screws do not come loose from the outer profile head 6 when being screwed in or loosened, since otherwise the screws could get into the person's body and cause significant damage there.

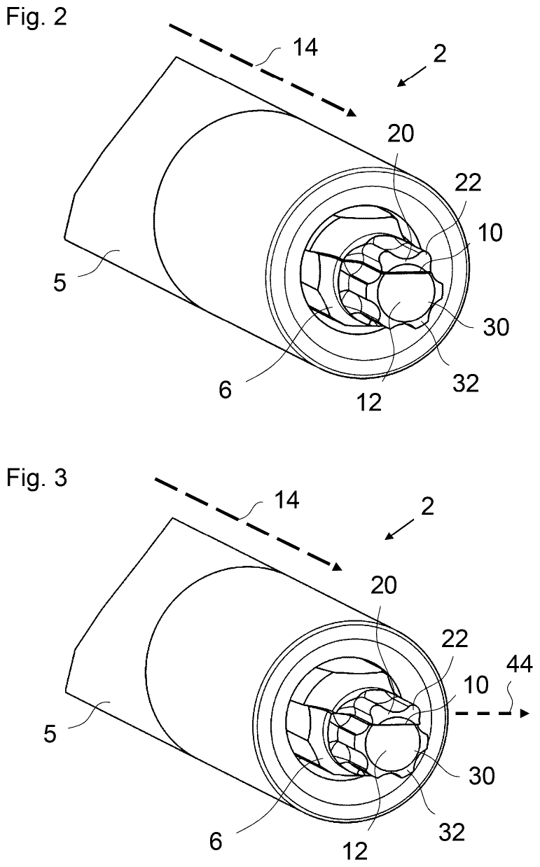

FIG. 2 shows, schematically simplified, an enlarged perspective representation of the outer profile head 6 at the distal end of the screwdriver 2 or rather of the shaft. The outer profile head 6 projects from the sleeve 5 in a longitudinal direction 14 of the screwdriver 2 or rather sleeve 5. The outer profile head 6 comprises an end profile 12 having multiple teeth 22, 32 in order to engage in the screw head drive of the screw. In the exemplary embodiment shown, the end profile 12 comprises six teeth 22, 32 and is provided with a star-shaped undulating profile, for example the profile known under the brand name “TORX®”, for screwing in screws. In order to clamp the outer profile head 6 in the screw head drive, the outer profile head 6 comprises a gap 10, which extends in the longitudinal direction 14 through the outer profile head 6. The gap 10 is arranged asymmetrically in the end profile 12, such that it splits the outer profile head 6 into a small profile head part 20 and a large profile head part 30. The small profile head part 20 comprises two teeth 22, whereas the large profile head part 30 has four teeth 32. For reasons of clarity, only one tooth 22, 32 has been provided with a reference sign in each case.

FIG. 3 shows, schematically simplified, the outer profile head 6 from FIG. 2 in a tensioned state. In this tensioned state, the small profile head part 20 is sheered with respect to the large profile head part 30 in a sheering direction 44. As a result, the outer profile head 6 can be clamped in the screw head drive of the screw and be securely held therein. The sheering direction 44 extends in a plane of the gap 10 parallel to the end profile 12.

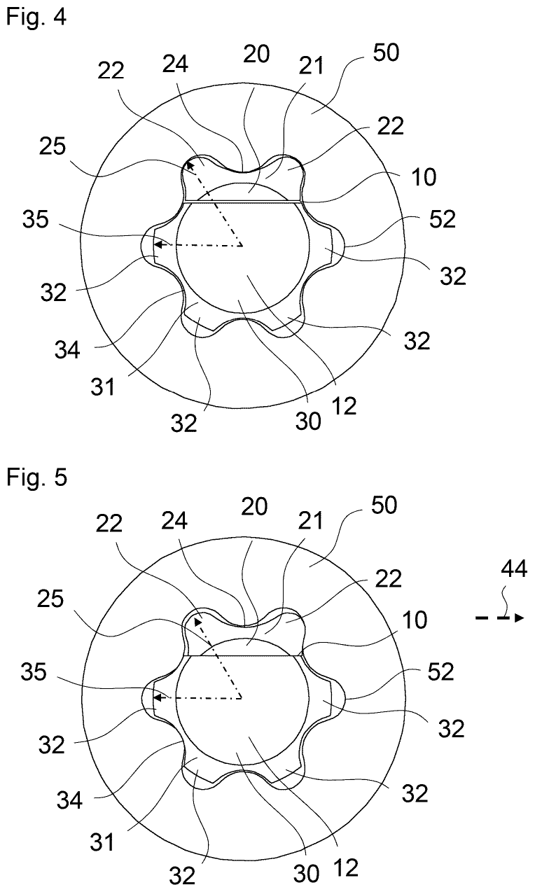

FIGS. 4 and 5 in each case show, schematically simplified, a cross-sectional view along the end profile 12 of the outer head profile 6, while the outer profile head 6 from FIG. 2 is arranged in a screw head drive 52 of an inner profile screw 50. In FIG. 4, the outer profile head 6 from FIG. 2 is shown in an untensioned state. In this representation, it is clear that the teeth 22 of the small profile head part 20 have a different shape to the teeth 32 of the large profile head part 30. Specifically, the teeth 22 project radially further out than the teeth 32, such that an end profile 21 of the small profile head part 20 has a greater outer radius 25 than the outer radius 35 of an end profile 31 of the large profile head part 30. The outer radii 25, 35 extend in each case from a center of the end profile 12 to a tip or rather a point on the teeth 22, 32 that protrudes furthest outward. In the present case, the teeth 22 of the small profile head part 20 are rounded, whereas the teeth 32 of the large profile head part 30 are flattened. For example, the teeth 22 have a profile known under the brand name “TORX®”, whereas the teeth 32 have a profile known under the brand name “TORX PLUS®”. This different design of the teeth 22, 32 has the effect that an end profile 21 of the small profile head part 20 fulfills a different function to an end profile 31 of the large profile head part 30. Specifically, the greater outer radius 25 of the teeth 22 ensures that the teeth 22 can be fixed well in the screw head drive 52 of the inner profile screw 50 and thus, during clamping, exert high holding forces which hold the outer profile head 6 from FIG. 3 against the inner profile screw 50. In contrast, the teeth 32 of the large profile head part 30 mainly have the function of absorbing the torques that arise when the inner profile screw 50 is tightened. On account of the flattened tips of the teeth 32, wear of the teeth 32 is significantly reduced. For this reason, it is advantageous to provide more teeth 32 than teeth 22, since in this way the teeth 32 absorb the majority of the arising torques. Outer profile edges 24, 34 which have the shape of a depression in the embodiment shown extend in each case between the teeth 32 and the teeth 22. In another embodiment (not shown), the outer profile edges may also have the shape of an outwardly curved circular arc, such that the end profile 12 overall has the shape of a circle from which the teeth 22, 32 protrude outward.

In FIG. 5, the end profile 12 and the inner profile screw 50 are shown in the tensioned state. It can be seen that the small profile head part 20 and the large profile head part 30 are sheered relative to one another in the sheering direction 44. As a result, the teeth 22 and 32 are fixed more firmly in the recesses of the screw head drive 52, such that the outer profile head 6 from FIG. 3 is clamped in the inner profile head screw 50. In the embodiment shown, the small profile head part 20, in addition to the movement in the sheering direction 44, is also pushed in the direction of the large profile head part 30, such that the gap 10 becomes smaller. According to other embodiments, the gap 10 can maintain the same width in the tensioned state as in the untensioned state without the profile head parts 20, 30 being moved toward one another.

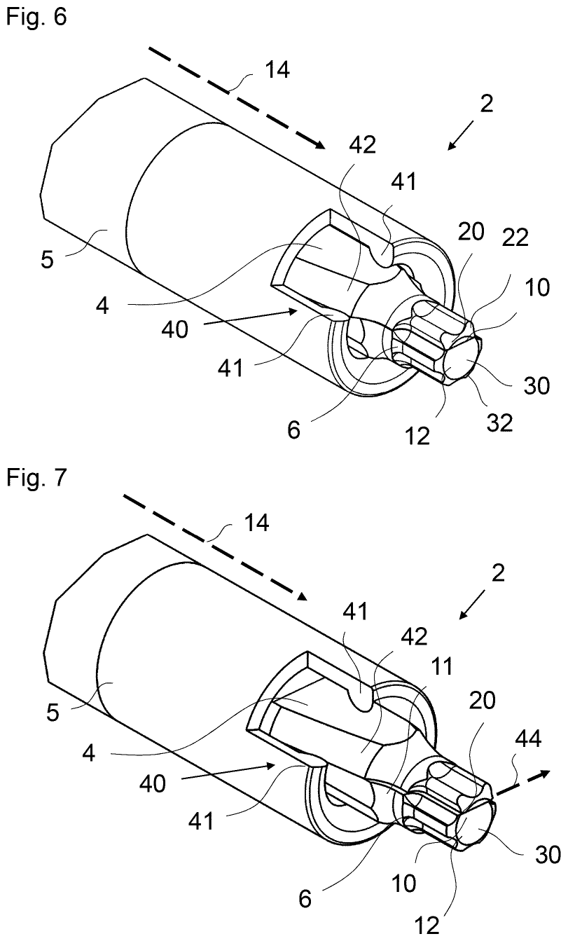

FIGS. 6 and 7 show the outer profile head 6 from FIGS. 2 and 3 in another perspective representation, in which part of the sleeve 5 has been cut out for better visibility of the inner components. This representation renders the shaft 4 visible, which extends in the sleeve 5. It can be seen that an outer surface of the shaft 4, which is located inside the sleeve 5, is designed as a run-on slope 42. Said run-on slope 42 is an inclined plane that slopes in the longitudinal direction 14. At the same time, the sleeve 5 comprises a run-on body 41, which is designed radially as a run-on ring or run-on ring segment in the exemplary embodiment shown. The run-on body 41 projects inward from the sleeve 5 and runs on the run-on slope 42. Due to the inclination of the run-on slope 42, this causes the run-on body 41 to push the run-on slope 42 and the small profile head part 20 connected thereto in the sheering direction 44 when the sleeve 5 is shifted counter to the longitudinal direction 14, as shown in FIG. 7. In this way, a clamping mechanism 40 is formed which comprises the run-on slope 42 and the run-on body 41.

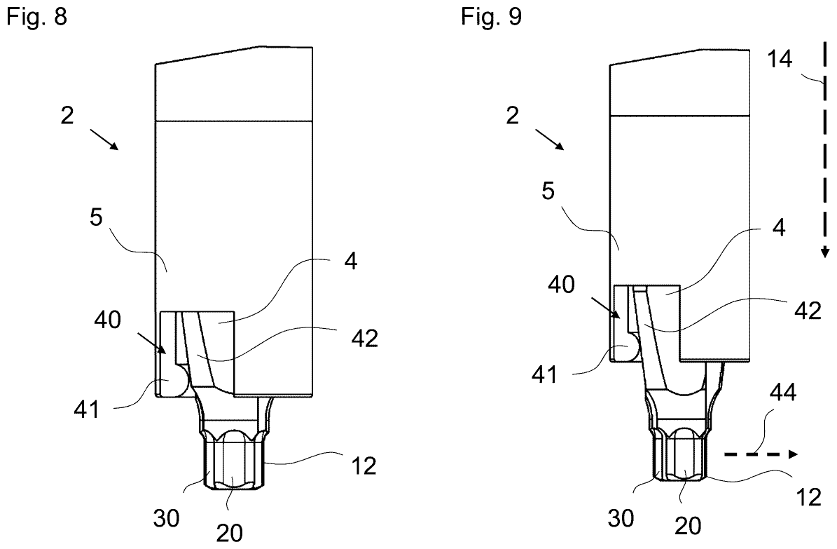

In FIGS. 8 and 9, the sleeve 5 and the outer profile head 6 from FIGS. 6 and 7 are shown in each case in a cross-sectional view, wherein FIG. 8 shows the untensioned state and FIG. 9 shows the tensioned state. In this view, it can clearly be seen how the run-on body 41 runs on the run-on slope 42 and thus moves the small profile head part 20 in the sheering direction 44 when the sleeve 5 is moved relative to the outer profile head 6 counter to the longitudinal direction 14. A movement of this kind of the sleeve 5 relative to the outer profile head 6 is brought about, for example, by a rotation of a component of the handle 8, of the like shown in FIG. 1. In this way, screws can be clamped on the outer profile head 6 or released again in a simple manner by means of the handle 8.

All of the indicated features, including those which are to be inferred from the drawings alone, and individual features which are disclosed in combination with other features, are deemed to be essential to the invention both alone and in combination. Embodiments according to the invention can be fulfilled by individual features or a combination of multiple features.

### LIST OF REFERENCE SIGNS

2 Screwdriver
4 Shaft
5 Sleeve
6 Outer profile head
7 Distal end
8 Handle
10 Gap
11 Cleavage plane
12 End profile
14 Longitudinal direction
20 Small profile head part
21 End profile
22 Tooth
24 Outer profile edge
25 Outer radius
30 Large profile head part
31 End profile
32 Tooth
34 Outer profile edge
35 Outer radius
40 Clamping mechanism
41 Run-on body
42 Run-on slope
44 Sheering direction
50 Inner profile screw
52 Screw head drive

What is claimed is:

1: A screwdriver for an inner profile screw, comprising:
a shaft; and
an outer profile head, which is arranged at a distal end of the screwdriver;
wherein the outer profile head comprises a gap along a longitudinal axis, which splits the outer profile head into a large profile head part and a small profile head part,
wherein a surface area of an end profile of the large profile head part is greater than a surface area of an end profile of the small profile head part,
wherein the end profile of the large profile head part and the end profile of the small profile head part in each case comprise multiple radially outwardly protruding teeth, and
wherein an outer radius of the end profile of the small profile head part is greater than an outer radius of the end profile of the large profile head part.

2: The screwdriver according to claim 1, wherein the teeth of the end profile of the large profile head part protrude less far radially outward than the teeth of the end profile of the small profile head part.

3: The screwdriver according to claim 1, wherein the teeth of the end profile of the small profile head part have rounded tips.

4: The screwdriver according to claim 1, wherein the end profile of the large profile head part comprises more teeth than the end profile of the small profile head part.

5: The screwdriver according to claim 1, wherein the end profile of the small profile head part and/or the end profile of the large profile head part is a star-shaped undulating profile.

6: The screwdriver according to claim 1, wherein the end profile of the small profile head part and/or the end profile of the large profile head part in each case comprise, between the teeth, outer profile edges that have the shape of a depression or an outwardly curved circular arc.

7: The screwdriver according to claim 6, wherein a circular arc through the points lying in each case centrally between two teeth on the outer profile edge of the small profile head part has the same circle radius as a circular arc through the points lying in each case centrally between two teeth on the outer profile edge of the large profile head part.

8: The screwdriver according to claim 1, wherein the end profile of the small profile head part and the end profile of the large profile head part have the same key size for screwing in screws with screw head drives of the same size.

9: The screwdriver according to claim 1, wherein a cleavage plane of the gap extends in a longitudinal direction of the shaft and in a direction parallel to the end profile of the profile head parts.

10: The screwdriver according to claim 1, wherein the gap splits the end profile of the outer profile head in an asymmetrical manner.

11: The screwdriver according to claim 1, further comprising a clamping mechanism for sheering the small profile head part and the large profile head part relative to one another, and wherein a sheering direction extends along the gap in parallel with the end profiles of the profile head parts.

12: The screwdriver according to claim 1, wherein the screwdriver comprises a sleeve that receives the outer profile head and that is configured to be screwed in with the outer profile head.

13: The screwdriver according to claim 12, wherein the outer profile head tapers toward the distal end in order to form at least one run-on slope, and wherein the sleeve comprises an inwardly protruding run-on body which is designed to run on the at least one run-up slope in order to sheer the small profile head part and the large profile head part relative to one another.
